(19) United States
(12) Patent Application Publication
(10) Pub. No.: US 2026/0083377 A1
(43) Pub. Date: Mar. 26, 2026

(54) ELECTROCARDIOGRAM ANALOG FRONT END

(71) Applicant: Bardy Diagnostics, Inc., Bellevue, WA (US)

(72) Inventor: Yunqiang LIN, Bellevue, WA (US)

(21) Appl. No.: 19/337,032

(22) Filed: Sep. 23, 2025

Related U.S. Application Data
(60) Provisional application No. 63/698,744, filed on Sep. 25, 2024.

(51) Int. Cl. A61B 5/308; A61B 5/00; A61B 5/256; A61B 5/257; A61B 5/28

(52) U.S. Cl. CPC A61B 5/308; A61B 5/256; A61B 5/257; A61B 5/28; A61B 5/6833; A61B 2560/0214; A61B 2560/0462

(57) ABSTRACT

An analog to digital converter for use in an electrocardiogram device is provided that includes a voltage supply; an operational amplifier, including: a positive input pin; an inverting input pin; and an output pin; an output node, connected directly to the output pin and the inverting input pin; an ECG electrode, connected to the output node via a first resistor; a reference voltage, connected directly to the output node; and a feedback node, connected to directly to the positive input pin, connected to the voltage supply via a second resistor, and connected to a ground via a third resistor and a first capacitor, wherein the third resistor and the first capacitor are connected in parallel between the feedback node and the ground.

### CROSS-REFERENCES TO RELATED DISCLOSURES

The present disclosure claims priority to U.S. Provisional Patent Application 63/698,744, filed on Sep. 25, 2024, titled “Electrocardiogram Analog Front End”, which is incorporated herein in its entirety.

### FIELD

The present disclosure generally relates to medical tools, and in particular to an analog front end (AFE) for use in electrocardiogram (ECG) signal gathering.

### BACKGROUND

Electrocardiograms (ECGs, also referred to as EKGs) are recordings of the electrical signals of a biological subject's heart. Electrocardiography measures the electrical activity of the cardiac cycle; graphing the voltage over time due to the muscle depolarization and repolarization that occurs during a heartbeat. Various medical conditions can be detected through the analysis of ECGs to identify shifts in rhythm, magnitude, or waveform shape from a baseline for the biological subject. An ECG in a human biological subject is measured from a baseline voltage and includes a series of waves (e.g., P-wave, Q-wave) that can be used to diagnose various cardiac conditions based on the relative magnitudes and timings in one heartbeat or across (whether consistently or sporadically) across several heartbeats.

### SUMMARY

The present disclosure provides enhanced electrocardiogram (ECG) analog front end (AFE). An ECG measurement patch adhered to the skin for the longitudinal analysis of a biological subject's ECG over an extended duration (e.g., a month or more) needs to be powered over the length of analysis period, which may require the replacement, recharging, or conservation of battery power in the device. As these devices are adhered to the skin of the biological subject, design goals frequently prioritize reducing power consumption, which reduces the size and weight of the needed battery and avoid the need to replace or recharge the battery, which in turn reduces the risk of an operator forgetting to replace/recharge the battery, improves adhesion to the biological subject's skin, reduces discomfort of wearing the devices, and various other benefits. However, conventional ECG AFEs often make sacrifices in one or more design goals, which may also include the responsiveness, accuracy, and cost of the devices used therein. Accordingly, conventional implementations offer suffer from one or more of high-power consumption, differential thermal noise, high noise (e.g., due to difference potential of the right-leg-drive (RLD) and the reference of the input stage, and complex designs with associated high costs and large sizes. Accordingly, the described devices are smaller and lighter and have a power source that can last over a longer duration that previous devices, among other benefits offered.

The design discussed in the present disclosure uses two operational amplifier (op-amp) based circuitries and two passive filter circuitries to achieve these and other advantages. Both op-amps can be low-cost general purpose op-amps. The first op-amp circuitry serves as input buffering, filtering, and amplifying stage.

In some embodiments, the first op-amp circuit is configured with a non-inverting op-amp, and may have a gain of 330, and a passband of 1-45 Hertz (Hz). The second op-amp circuitry serves as common mode drive and direct current (DC) cancelation, and may be configured as integrator with 0 decibels (dB) at a frequency of 0.007 Hz. This pole together with the first op-amp forms a closed-loop feedback circuit, which results in a high pass filter of 0.007 Hz. The two op-amps interface with two electrodes respectively. The first op-amp is configured as noninverting with DC coupling from the first electrode. The second op-amp is configured as integrator using the first op-amp's output as its input, and drive to the second electrode. Before the first op-amp, a passive lowpass filter consists of protection resistor and shunting capacitor. This arrangement takes the advantage of protection resistor to filter high frequency content (e.g., ˜9 kHz or higher). After the two-op-amp-based closed loop, an additional passive anti-aliasing filter can be added at the trade-off of extra power consumption.

In some embodiments, the first op-amp can be configured with capacitive feedback instead of resistive feedback. Capacitive feedback offers better passband flatness at the trade-off of corner frequency accuracy. Because of better granularity of resistors available, resistive feedback offers better performance adjusting and component flexibility than capacitive feedback.

In some embodiments, a third op-amp configured as inverter with unity gain can be added to invert the second op-amp's output and apply that inverted output to the first op-amp's feedback reference. This arrangement significantly improves the transient response at the trade-off of the additional op-amp circuitry.

In some embodiments, the second op-amp can be split into two op-amp-based circuitries: a first that serves a unity gain common mode drive, a second that cancels the DC offset. This arrangement improves the transient response, reduces the sensitivity to skin-electrode impedance, at the trade-off of the additional op-amp circuitry.

Each of the described embodiments may add the capability of sensing the feedback of the second op-amp. This additional sensing allows aggressive transient response, wider dynamic range at low frequency, at the trade-off of additional ADC channel data storage.

In one embodiment, an analog front end for an electrocardiogram (ECG) measuring device is provided, including: a first op-amp, configured to receive a signal input from an ECG patch at a first input port and a feedback signal at a second input port and to produce an amplified output of an ECG signal via an output port; and a second op-amp, configured as an integrator that receives the amplified output from the first op-amp at a second input port and integrates the amplified output against a common voltage received at a first input port to produce an integrated output via an output port that is provided as a reference output and as at least a portion of the feedback signal.

In some such embodiments, the analog front end further includes: a resistive feedback loop, connected between the output port of the first op-amp and the second input port of the first op-amp that provides the amplified output as at least a portion of the feedback signal.

In some such embodiments, the analog front end further includes: a capacitive feedback loop, connected between the output port of the first op-amp and the second input port of the first op-amp that provides the amplified output as at least a portion of the feedback signal.

In some such embodiments, the analog front end further includes: an integrator feedback loop, including a capacitor connected between the output port of the second op-amp and the second input port of the second op-amp that provides the reference output as a second feedback signal to the second input port of the second op-amp.

In some such embodiments, the analog front end further includes: a capacitor connected between the first input port of the first op-amp and the second input port of the first op-amp.

In some such embodiments, the analog front end further includes: a passive lowpass filter connected between an input for the ECG patch and a output for the reference output.

In some such embodiments, the analog front end further includes: a third op-amp, configured as a unity integrator that receives the amplified output from the first op-amp and the reference output from the second op-amp at a second input port and integrates the amplified output and the reference output as combined against the common voltage received at a first input port to produce an inverted integrated output via an output port that is provided as a component of the reference output.

In some such embodiments, the analog front end further includes: an inverted integrator feedback loop, including a resistor connected between the output port of the third op-amp and the second input port of the third op-amp that provides the inverted integrated output as a third feedback signal to the second input port of the third op-amp.

In one embodiment, an analog front end is provided including: a first op-amp, having a first input port, a second input port and a first output port; and a second op-amp, having a third input port, a fourth input port, and a second output port, wherein: a common voltage source is in communication with the third input port; the first output port is in communication with the fourth input port; and the first output port and the second output port are in communication with the second input port.

In some such embodiments, the analog front end, further includes: an electrocardiogram (ECG) patch in communication with the first input port and the second output port; and an electrocardiogram in communication with the first output port.

In some such embodiments, the analog front end, further includes: the first input port and the third input port are non-inverting and the second input port and the fourth input port are inverting.

In some such embodiments, the analog front end, further includes: a resistive feedback loop, connected between the first output port and the second input port.

In some such embodiments, the analog front end, further includes: a capacitive feedback loop, connected between the first output port and the second input port.

In some such embodiments, the analog front end, further includes: an integrator feedback loop, including a capacitor connected between the second output port of the second op-amp and the fourth input port.

In some such embodiments, the analog front end, further includes: a capacitor connected between the first input port and the second input port.

In some such embodiments, the analog front end, further includes: a third op-amp, having a fifth input port, a sixth input port, and a third output port, wherein: the common voltage source is in communication with the fifth input port; the sixth input port is in communication with the first output port and the second output port; and the third output port is in communication with the second output port.

In some such embodiments, the analog front end, further includes: an inverted integrator feedback loop, including a resistor connected between the third output port and the sixth input port.

In some such embodiments, the analog front end, the second output port is in communication with the second input port via an integrated refence feedback line that includes one or both of a resistor and a capacitor in series between the second output port and the second input port.

In one embodiment, a wearable electrocardiography monitor is provided that includes an extended wear electrode patch including a battery and an analog front end, the analog front end including: a first op-amp, having a first input port, a second input port and a first output port; and a second op-amp, having a third input port, a fourth input port, and a second output port, wherein: the battery is in communication with the third input port; the first output port is in communication with the fourth input port; and the first output port and the second output port are in communication with the second input port.

In some such embodiments, the analog front end further includes: a third op-amp, having a fifth input port, a sixth input port, and a third output port, wherein: the battery is in communication with the fifth input port; the sixth input port is in communication with the first output port and the second output port; and the third output port is in communication with the second output port.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, including time and clustering of events, all without departing from their spirit and the scope. Accordingly, the drawings and detailed description are to be regarded as illustrative in nature and not as restrictive.

### BRIEF DESCRIPTION OF THE DRAWINGS

FIG. 1 illustrates an example of an electrocardiography monitor, including an extended wear electrode patch fitted to the sternal region of a human male biological subject, according to embodiments of the present disclosure.

FIGS. 2A and 2B are circuit diagrams of an example circuit layout for an electrocardiogram (ECG) analog front end (AFE), according to embodiments of the present disclosure.

FIG. 3 is a circuit diagram of an example power controller for an ECG AFE, according to embodiments of the present disclosure.

FIG. 4 illustrates digital signals used by the ECG monitor described herein for measuring an ECG waveform, according to embodiments of the present disclosure.

FIG. 5 illustrates an example computing device, according to embodiments of the present disclosure.

### DETAILED DESCRIPTION

The present disclosure provides enhanced electrocardiogram (ECG) analog front end (AFE). An ECG measurement patch adhered to the skin for the longitudinal analysis of a biological subject's ECG over an extended duration (e.g., a month or more) needs to be powered over the length of analysis period, which may require the replacement, recharging, or conservation of battery power in the device. As these devices are adhered to the skin of the biological subject, design goals frequently prioritize reducing power consumption, which reduces the size and weight of the needed battery and avoid the need to replace or recharge the battery, which in turn reduces the risk of an operator forgetting to replace/recharge the battery, improves adhesion to the biological subject's skin, reduces discomfort of wearing the devices, and various other benefits. However, conventional ECG AFEs often make sacrifices in one or more design goals, which may also include the responsiveness, accuracy, and cost of the devices used therein. Accordingly, conventional implementations offer suffer from one or more of high-power consumption, differential thermal noise, high noise (e.g., due to difference potential of the right-leg-drive (RLD) and the reference of the input stage, and complex designs with associated high costs and large sizes. Accordingly, the described devices are smaller and lighter and have a power source that can last over a longer duration that previous devices, among other benefits offered.

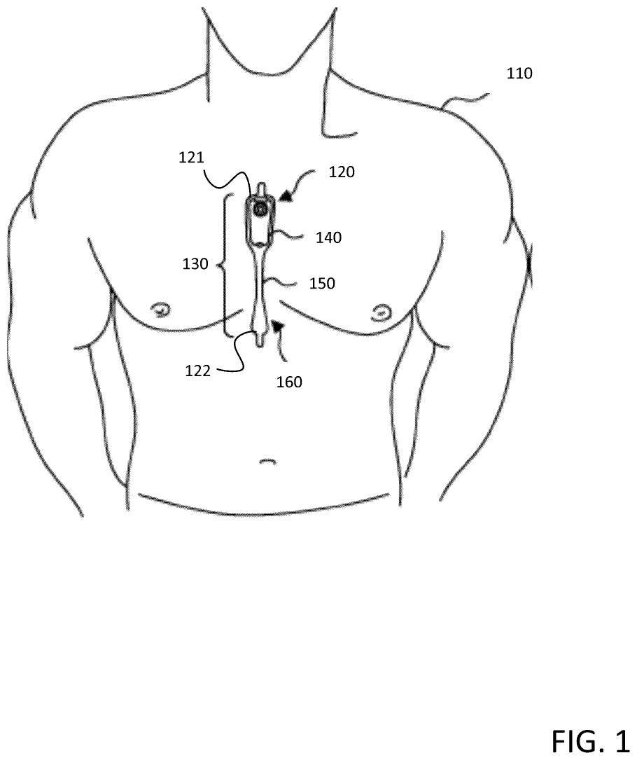

FIG. 1 illustrates an example of an electrocardiography monitor, including an extended wear electrode patch fitted to the sternal region of a human male biological subject, according to embodiments of the present disclosure. ECG and physiological monitoring can be provided through a wearable ambulatory monitor that includes two components, a flexible extended wear electrode patch and a removable reusable (or single use) monitor recorder. Both the electrode patch and the monitor recorder are configured to capture electrical signals from the propagation of low amplitude, relatively low frequency content cardiac action potentials, such as the P-waves generated during atrial activation. By way of example, FIG. 1 shows an extended wear electrocardiography monitor 120, including a monitor recorder 140. The wearable monitor 120 sits centrally, positioned axially along the sternal midline 160, on the subject's chest along the sternum 130 and oriented top-to-bottom with the monitor recorder 140 situated (preferably) towards the subject's head.

The electrode patch 150 is shaped to fit comfortably and conformal to the contours of the subject's chest approximately centered on the sternal midline 160 (or immediately to either side of the sternum 130). The distal end of the electrode patch 150, under which a lower or inferior pole 122 (ECG electrode) is adhered, extends towards the Xiphoid process and lower sternum, and (depending upon the subject's build) may straddle the region over the Xiphoid process and lower sternum. The proximal end of the electrode patch 150, located under the monitor recorder 140, under which an upper or superior pole 121 (ECG electrode) is adhered, is below the manubrium and, depending upon subject's build, may straddle the region over the manubrium.

Although discussed herein primarily with reference to a biological subject that is human, the present disclosure contemplates that the teachings provided herein may be applied for electrocardiographic devices used with non-human biological subjects. Accordingly, the various anatomical terms used to describe physiological features and relative locations in a human model may be modified to describe recognizably similar physiological features and relative locations in non-human models.

During ECG monitoring, the amplitude and strength of action potentials sensed on the body's surface are affected to varying degrees by cardiac, cellular, extracellular, vector of current flow, and physical factors, like obesity, dermatitis, high impedance skin, etc. Sensing along the sternal midline 160 (or immediately to either side of the sternum 130) significantly improves the ability of the wearable monitor 120 to cutaneously sense cardiac electric signals, particularly the P-wave (or atrial activity) and, to a lesser extent, the QRS interval signals in the ECG waveforms that indicate ventricular activity by countering some of the effects of these factors. Depending upon their placement locations on the chest, ECG electrodes may be separated from activation regions within the heart by differing combinations of internal tissues and body structures, including heart muscle, intracardiac blood, the pericardium, intrathoracic blood and fluids, the lungs, skeletal muscle, bone structure, subcutaneous fat, and the skin, plus any contaminants present between the skin's surface and electrode signal pickups. The degree of amplitude degradation of cardiac transmembrane potentials increases with the number of tissue boundaries between the heart and the skin's surface that are encountered. The cardiac electrical field is degraded each time the transmembrane potentials encounter a physical boundary separating adjoining tissues due to differences in the respective tissues' electrical resistances. In addition, other non-spatial factors, such as pericardial effusion, emphysema or fluid accumulation in the lungs, can further degrade body surface potentials.

These degradations in the heartbeat signal due to effects from the physiology of the biological subject all decrease the Signal to Noise Ratio (SNR) in generating an ECG; either by increasing noise magnitude or decreasing signal magnitude.

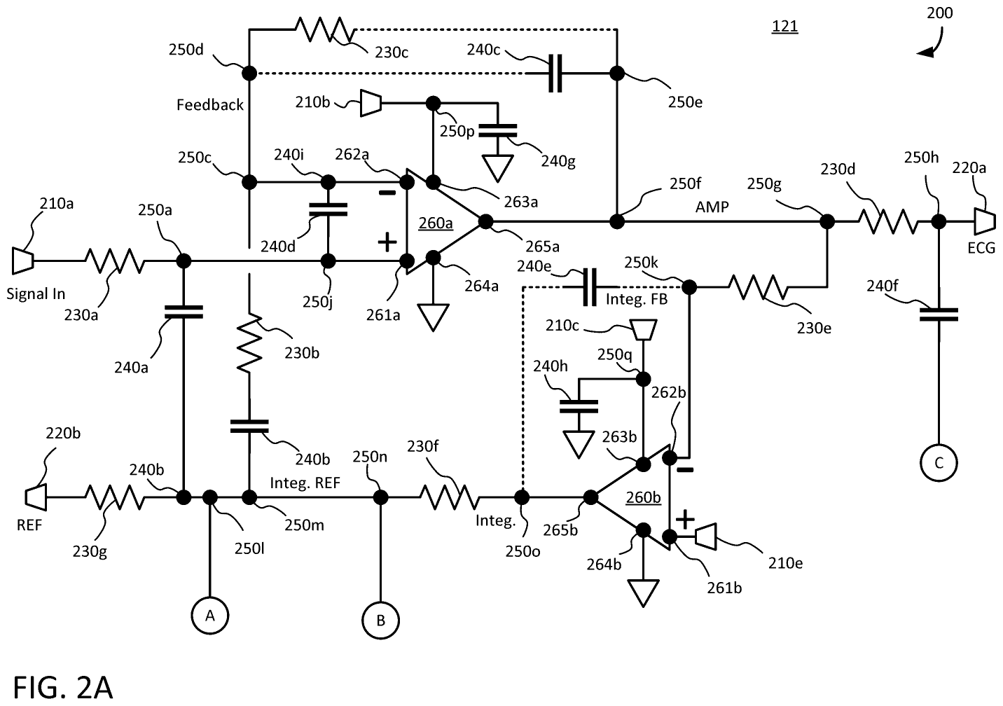

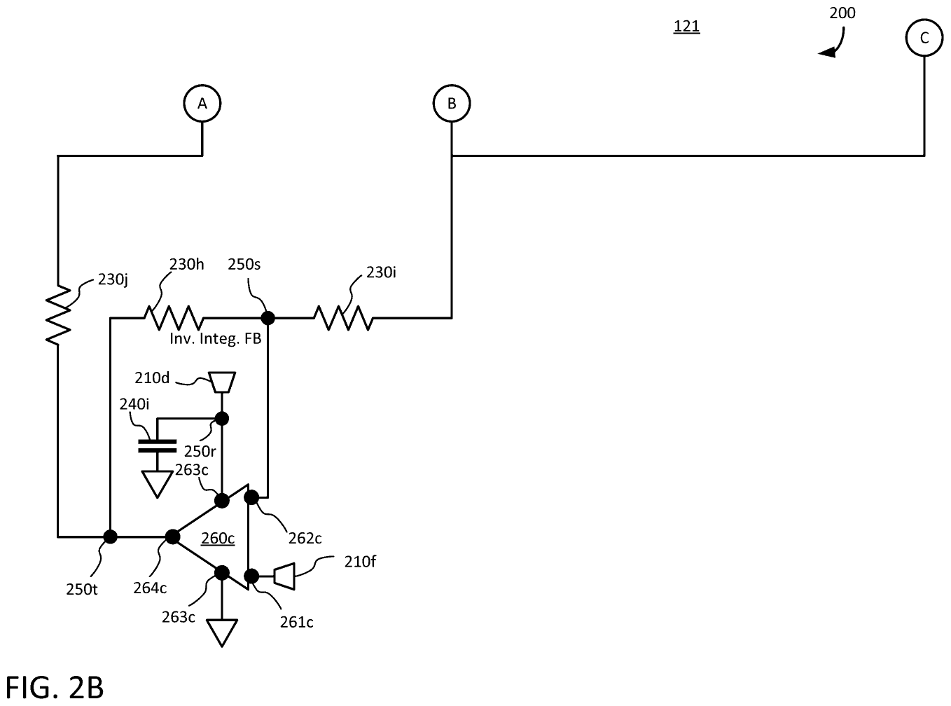

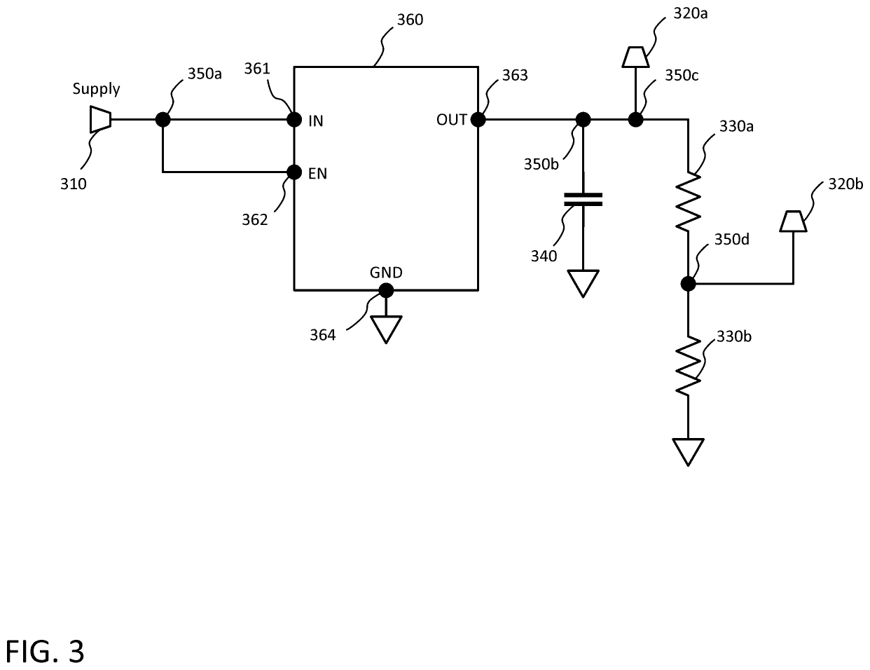

As is discussed in greater detail in regards to FIGS. 2A-2B and 3, the wearable monitor 120 may be understood as having two patch connections that operate together to provide the functionalities described herein. The first patch connection is primarily the ECG analog front end, which receives an input from an electrode near or connected to the inferior pole 122. The input is connected to an electrode near the inferior pole 122. The output of the first patch 150 is filtered to produce a low frequency rejected ECG signal that goes to an Analog-to-Digital Converter in the Micro Control Unit (MCU).

The second patch connection describes the driving circuitry that drives the second electrode that is near or connected to the superior pole 121. This driving circuitry drives the second electrode at the midpoint of the analog frontend supply, and ultimately serves as a reference signal for the first patch connection (e.g., output 220b).

Both patches may use the same battery and ground, and may be constructed on the same printed circuit board as one another. As part of self-testing, the MCU drives the second patch with a predefined voltage/current pattern and that drives a signal pass through the body of a biological subject wearing the wearable monitor 120, which is sensed as part of the ECG signal through the first patch.

FIGS. 2A and 2B are circuit diagrams of an example circuit topology for an electrocardiogram (ECG) analog front end (AFE), as may be used in a first patch connection 200, according to embodiments of the present disclosure. In various embodiments, the first patch connection 200 is located in or in electrical communication with a superior pole 121 of a wearable monitor 120, such as the example shown in FIG. 1, that is positioned superior to the xiphoid process of the subject 110 and inferior to or proximal to the manubrium of the subject 110. In some embodiments, some or all of the electrical components of the first patch connection 200 are located in a monitor recorder 140 of the wearable monitor 120.

In FIGS. 2A-2B, various components are designated by like numerals and unique letter designators to distinguish, the various components from one another, but not all components may be included in every embodiment. Accordingly, although first through sixth inputs 210a-f (generally or collectively, inputs 210), first and second outputs 220a-b (generally or collectively, outputs 220), first through tenth resistors 230a-j (generally or collectively, resistors 230), first through ninth capacitors 240a-i (generally or collectively, capacitors 240), first through twentieth nodes 250a-t (generally or collectively, nodes 250), a first through third operational amplifiers (op-amps) 260a-c are illustrated in FIGS. 2A and 2B, in some embodiments some of the listed components may be omitted or reconfigured. For example, the components illustrated in FIG. 2B may be omitted from the AFE in some embodiments (e.g., by disconnecting the ninth and tenth resistors 230i-j or setting the resistance thereof to be infinite). In another example, one of the third resistor 230c or the third capacitor 240c may be omitted (and the other included) to provide resistive or capacitive feedback for the first op-amp 260a.

Each of the op-amps 260a-c is illustrated with a correspondingly designated set of ports, with a first input port 261a-c, a second input port 262a-c, a positive reference port 263a-c, a negative reference port 264a-c, and an output port 265a-c. Additionally, each negative reference port 264a-c is grounded, and each positive reference port 263a-c is connected to a node 250p-r connected to a voltage inputs 210b-d and to a capacitor 240g-i that is connected to ground. In various embodiments, the voltage inputs 210b-d may supply a 2.5 Volt (V) Direct Current (DC) power to the respective op-amp 260a-c, and the respective capacitors 240g-i may have a capacitance of 0.1 microfarads (μF) to provide power supply filtering to the positive ports 263a-c. In various embodiments, each of the groundings may refer to the same ground.

The illustrated design may use the first op-amp 260a and the second op-amp 260b with associated passive filter circuitries (e.g., omitting the third op-amp 260a and associated circuitry), or may use all three op-amps 260a-c and associated circuitry.

The first op-amp 260a and associated circuitry serves as input buffering, filtering, and amplifying stage for the overall design. In some embodiments, the first op-amp circuit is configured with a non-inverting op-amp 260a, and may have a gain of 330, and a passband of 1-45 Hertz (Hz). Before the first op-amp 260a, a passive lowpass filter consists of protection resistor 230a and shunting capacitor 240a. This arrangement takes the advantage of protection resistor 230a to filter out high frequency content (e.g., ˜9 kHz or higher). In various embodiments, a capacitor 240d is disposed between the first port 261a and the second port 262a. In various embodiments, the second port 262a of the first op-amp 260a may receives feedback signals from various outputs of the op-amps 260, each of which may form a portion or all of the feedback signal provided to the first op-amp 260a.

In various embodiments, the first op-amp 260a can be configured with one of capacitive feedback (e.g., via capacitor 240c) or resistive feedback (e.g., via resistor 230c) between nodes 250d-e. Capacitive feedback offers better passband flatness at the trade-off of corner frequency accuracy relative to resistive feedback. Because of better granularity of resistors available, resistive feedback offers better performance adjusting and component flexibility than capacitive feedback.

The second op-amp 260b and associated circuitry serves as common mode drive and DC cancelation, and may be configured as integrator with 0 decibels (dB) at a frequency of 0.007 Hz. This pole together with the first op-amp 260a forms a closed-loop feedback circuit, which results in a high pass filter of 0.007 Hz. The two op-amps 260a-b interface with two electrodes respectively. The first op-amp 260a is configured as noninverting with DC coupling from the first input 210a (e.g., the signal in from the patch 150). The second op-amp 260b is configured as an integrator using the output from the first op-amp 260a output (e.g., from port 265a) as an input to the second input port 262b, which drives to the electrode for the second output 220b. In various embodiments, each of the second op-amp 260b and the (optional) third op-amp 260c are provided with an input signal 210e or 210f at a respective first port 261c of a common voltage (e.g., 1.25 V or half of the voltage applied via the second, third, and fourth inputs 230b-d).

After the closed loop formed by the two op-amps 260a-b, an additional passive anti-aliasing (lowpass) filter can optionally be added at the trade-off of extra power consumption.

In some embodiments, a third op-amp 260c configured as inverter with unity gain can be added to invert the output from the second op-amp 260b, and apply that inverted output to the feedback reference for the first op-amp 260a (e.g., at node 2501). This arrangement significantly improves the transient response at the trade-off of the additional op-amp circuitry. In various embodiments, the integrated refence feedback (e.g., from node 250n to node 250c) similarly to the amplified feedback (e.g., from node 250f to node 250d) may be resistive (include second resistor 230b) or capacitive (include second capacitor 240b), or may be both resistive and capacitive (e.g., include both the second resistor 230b and the second capacitor 240b).

In some embodiments, the second op-amp 260b can be split into two op-amp based circuitries: a first that serves a unity gain common mode drive, a second that cancels the DC offset. This arrangement improves the transient response, reduces the sensitivity to skin-electrode impedance, at the trade-off of the additional op-amp circuitry.

Each of the described embodiments may add the capability of sensing the feedback of the second op-amp 260b. This additional sensing allows aggressive transient response, wider dynamic range at low frequency, at the trade-off of additional ADC channel data storage.

In various embodiments, each of the components may have about the following values.

TABLE 1
Component Reference Value

| Component | Value | |
|---|---|---|
| Input 210a (Signal) | Variable | |
| Input 210b (Driving) | 2.5 | V |
| Input 210c (Driving) | 2.5 | V |
| Input 210d (Common) | 1.25 | V |
| Input 210e (Common) | 1.25 | V |
| Input 210f (Driving) | 2.5 | V |
| Resistor 230a | 68.1 | kΩ |
| Resistor 230b | 30 kΩ or 0 Ω | |
| Resistor 230c | 10 MΩ or 0 Ω | |
| Resistor 230d | 330 | kΩ |
| Resistor 230e | 10 | MΩ |
| Resistor 230f | 0 | Ω |
| Resistor 230g | 0 | Ω |
| Resistor 230h | 10 | kΩ |
| Resistor 230i | 10 kΩ or ∞ Ω | |
| Resistor 230j | 0 Ω or ∞ Ω | |
| Capacitor 240a | 100 | pF |
| Capacitor 240b | 10 | μF |
| Capacitor 240c | 330 pF or 0 F | |
| Capacitor 240d | 10 | pF |
| Capacitor 240e | 4.7 | μF |
| Capacitor 240f | 10 | nF |
| Capacitor 240g | 0.1 | μF |
| Capacitor 240h | 0.1 | μF |
| Capacitor 240i | 0.1 | μF |

FIG. 3 is a circuit diagram of an example power controller 300 for an ECG AFE, according to embodiments of the present disclosure. In various embodiments, a microprocessor or other voltage regulator 360 is provided control the driving voltage and common voltage supplied to the ECG AFE 200 discussed in relation to FIGS. 2A and 2B. A DC supply voltage input 310 is provided from a battery to an input pin 361 and an enable pin 362 to yield an output voltage at an output port 363. The regulator 360 is also connected to ground via a ground port 364.

The output from the regulator 360 provides a first regulated output 320a of the driving voltage and a second regulated output 320b of the common voltage used by the op-amps 260. A first resistor 330a and a second resistor 330b provide a voltage splitter that halves the driving voltage to produce the common voltage at a fourth node 350d between the resistors 330a-b. In various embodiments, each of the first and second resistors 330a-b are 1 MΩ.

In some embodiments, a capacitor 340 is disposed between a second node 350b and ground, where the second node 350b is disposed between the output port 363 of the regulator 360 and a third node 350c where the driving voltage is drawn, to provide for high-frequency filtering. In various embodiments, the capacitor 340 is 2.2 μF.

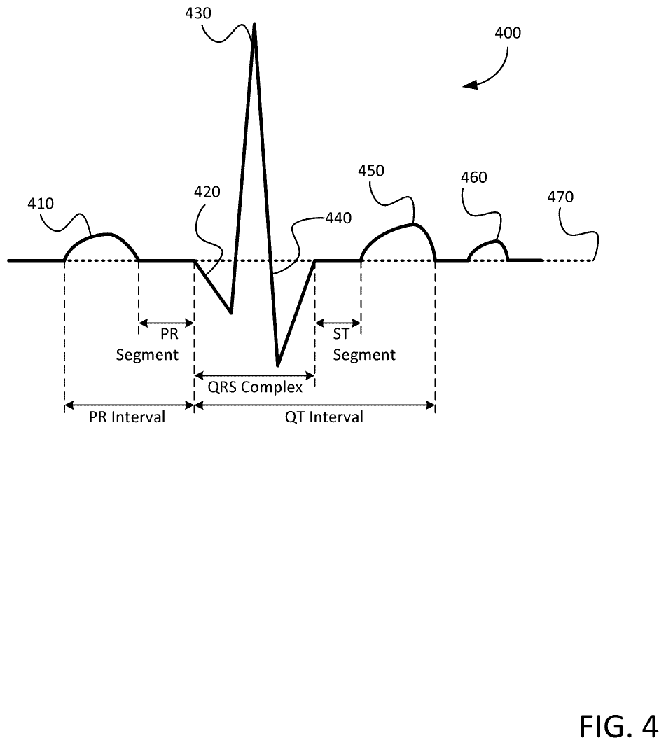

FIG. 4 illustrates components of an example ECG waveform 400, on which the x-axis represents time, and the y-axis represents cutaneous electrical signal strength relative to a baseline 470. The P-wave 410 has a smooth, normally upward, that is, positive, waveform that indicates atrial depolarization. The QRS complex often begins with the downward deflection of a Q-wave 420, followed by a larger upward deflection of an R-wave 430, and terminated with a downward waveform of the S-wave 440, collectively representative of ventricular depolarization. The T-wave 450 is normally a modest upward waveform, representative of ventricular depolarization, while the U-wave 460, often not directly observable, indicates the recovery period of the Purkinje conduction fibers.

Sampling of the intervals between successive waveforms 400 enables heart rate information derivation. For instance, the R-to-R interval represents the ventricular rate and rhythm, while the P-to-P interval represents the atrial rate and rhythm. Importantly, the PR interval is indicative of atrioventricular (AV) conduction time and abnormalities in the PR interval can reveal underlying heart disorders, thus representing another reason why the P-wave quality achievable by the ambulatory electrocardiography monitoring patch optimized for capturing low amplitude cardiac action potential propagation described herein is medically unique and important. The long-term observation of these ECG indicia, as provided through extended wear of the wearable monitor 120, provides valuable insights to the subject's cardiac function symptoms, and overall well-being, which may be used for the treatment or prophylaxis of various conditions.

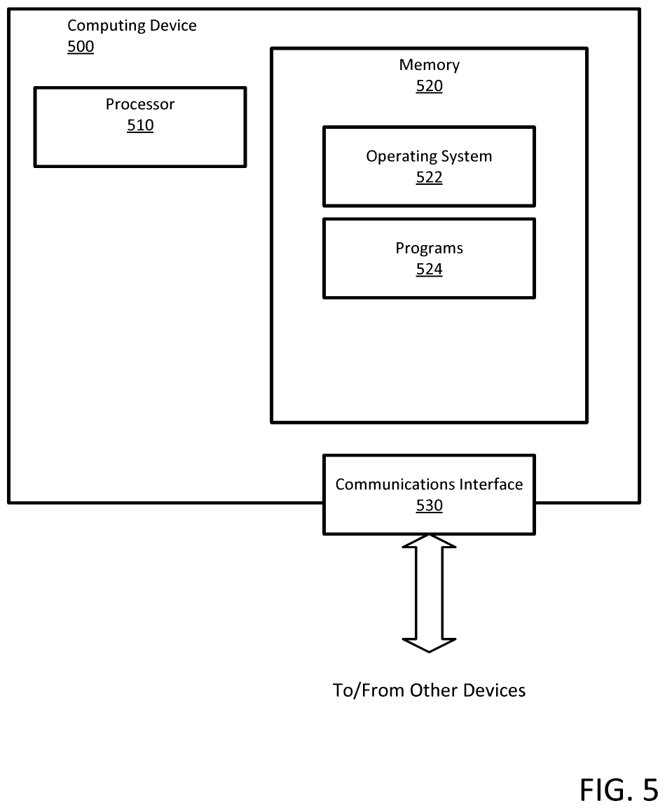

FIG. 5 illustrates a computing device 500, as may be used as an MCU to collect, collate, and output digital measurements of an ECG signal to produce an ECG, according to embodiments of the present disclosure. The computing device 500 may include at least one processor 510, a memory 520, and a communication interface 530.

The processor 510 may be any processing unit capable of performing the operations and procedures described in the present disclosure. In various embodiments, the processor 510 can represent a single processor, multiple processors, a processor with multiple cores, and combinations thereof.

The memory 520 is an apparatus that may be either volatile or non-volatile memory and may include RAM, flash, cache, disk drives, and other computer readable memory storage devices. Although shown as a single entity, the memory 520 may be divided into different memory storage elements such as RAM and one or more hard disk drives. As used herein, the memory 520 is an example of a device that includes computer-readable storage media, and is not to be interpreted as transmission media or signals per se.

As shown, the memory 520 includes various instructions that are executable by the processor 510 to provide an operating system 522 to manage various features of the computing device 500 and one or more programs 524 to provide various functionalities to users of the computing device 500, which include one or more of the features and functionalities described in the present disclosure. One of ordinary skill in the relevant art will recognize that different approaches can be taken in selecting or designing a program 524 to perform the operations described herein, including choice of programming language, the operating system 522 used by the computing device 500, and the architecture of the processor 510 and memory 520. Accordingly, the person of ordinary skill in the relevant art will be able to select or design an appropriate program 524 based on the details provided in the present disclosure.

The communication interface 530 facilitates communications between the computing device 500 and other devices, which may also be computing devices as described in relation to FIG. 5. In various embodiments, the communication interface 530 includes antennas for wireless communications and various wired communication ports. The computing device 500 may also include or be in communication, via the communication interface 530, one or more input devices (e.g., a keyboard, mouse, pen, touch input device, etc.) and one or more output devices (e.g., a display, speakers, a printer, etc.). The computing device 500 may be in communication with one or more of the patch connection 200 and power controller 300.

Although not explicitly shown in FIG. 5, it should be recognized that the computing device 500 may be connected to one or more public and/or private networks via appropriate network connections via the communication interface 530. It will also be recognized that software instructions may also be loaded into a non-transitory computer readable medium such as the memory 520 from an appropriate storage medium or via wired or wireless means.

Accordingly, the computing device 500 is an example of a system that includes a processor 510 and a memory 520 that includes instructions that (when executed by the processor 510) perform various embodiments of the present disclosure. Similarly, the memory 520 is an apparatus that includes instructions that when executed by a processor 510 perform various embodiments of the present disclosure.

Certain terms are used throughout the description and claims to refer to particular features or components. As one skilled in the art will appreciate, different persons may refer to the same feature or component by different names. This document does not intend to distinguish between components or features that differ in name but not function.

As used herein, various units of measure may be referred to by associated short forms with various prefixes applied thereto as set by the International System of Units (SI), which one of ordinary skill in the relevant art will be familiar with.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the present description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

While the invention has been particularly shown and described as referenced to the embodiments thereof, those skilled in the art will understand that the foregoing and other changes in form and detail may be made therein without departing from the spirit and scope.

What is claimed is:

1. An analog front end for an electrocardiogram (ECG) measuring device, comprising:
a first op-amp, configured to receive a signal input from an ECG patch at a first input port and a feedback signal at a second input port and to produce an amplified output of an ECG signal via an output port; and
a second op-amp, configured as an integrator that receives the amplified output from the first op-amp at a second input port and integrates the amplified output against a common voltage received at a first input port to produce an integrated output via an output port that is provided as a reference output and as at least a portion of the feedback signal.

2. The analog front end of claim 1, further comprising:
a resistive feedback loop, connected between the output port of the first op-amp and the second input port of the first op-amp that provides the amplified output as at least a portion of the feedback signal.

3. The analog front end of claim 1, further comprising:
a capacitive feedback loop, connected between the output port of the first op-amp and the second input port of the first op-amp that provides the amplified output as at least a portion of the feedback signal.

4. The analog front end of claim 1, further comprising:
an integrator feedback loop, including a capacitor connected between the output port of the second op-amp and the second input port of the second op-amp that provides the reference output as a second feedback signal to the second input port of the second op-amp.

5. The analog front end of claim 1, further comprising:
a capacitor connected between the first input port of the first op-amp and the second input port of the first op-amp.

6. The analog front end of claim 1, further comprising:
a passive lowpass filter connected between an input for the ECG patch and a output for the reference output.

7. The analog front end of claim 1, further comprising:
a third op-amp, configured as a unity integrator that receives the amplified output from the first op-amp and the reference output from the second op-amp at a second input port and integrates the amplified output and the reference output as combined against the common voltage received at a first input port to produce an inverted integrated output via an output port that is provided as a component of the reference output.

8. The analog front end of claim 7, further comprising:
an inverted integrator feedback loop, including a resistor connected between the output port of the third op-amp and the second input port of the third op-amp that provides the inverted integrated output as a third feedback signal to the second input port of the third op-amp.

9. An analog front end, comprising:
a first op-amp, having a first input port, a second input port and a first output port; and
a second op-amp, having a third input port, a fourth input port, and a second output port,
wherein:
a common voltage source is in communication with the third input port;
the first output port is in communication with the fourth input port; and
the first output port and the second output port are in communication with the second input port.

10. The analog front end of claim 9, further comprising:
an electrocardiogram (ECG) patch in communication with the first input port and the second output port; and
an electrocardiogram in communication with the first output port.

11. The analog front end of claim 9, wherein the first input port and the third input port are non-inverting and the second input port and the fourth input port are inverting.

12. The analog front end of claim 9, further comprising:
a resistive feedback loop, connected between the first output port and the second input port.

13. The analog front end of claim 9, further comprising:
a capacitive feedback loop, connected between the first output port and the second input port.

14. The analog front end of claim 9, further comprising:
an integrator feedback loop, including a capacitor connected between the second output port of the second op-amp and the fourth input port.

15. The analog front end of claim 9, further comprising:
a capacitor connected between the first input port and the second input port.

16. The analog front end of claim 9, further comprising:
a third op-amp, having a fifth input port, a sixth input port, and a third output port,
wherein:
the common voltage source is in communication with the fifth input port;
the sixth input port is in communication with the first output port and the second output port; and
the third output port is in communication with the second output port.

17. The analog front end of claim 16, further comprising:
an inverted integrator feedback loop, including a resistor connected between the third output port and the sixth input port.

18. The analog front end of claim of claim 9, wherein the second output port is in communication with the second input port via an integrated refence feedback line that includes one or both of a resistor and a capacitor in series between the second output port and the second input port.

19. A wearable electrocardiography monitor, comprising:
an extended wear electrode patch including a battery and an analog front end, the analog front end including:
a first op-amp, having a first input port, a second input port and a first output port; and
a second op-amp, having a third input port, a fourth input port, and a second output port,
wherein:
the battery is in communication with the third input port;
the first output port is in communication with the fourth input port; and
the first output port and the second output port are in communication with the second input port.

20. The wearable electrocardiography monitor of claim 19, wherein the analog front end further includes:
a third op-amp, having a fifth input port, a sixth input port, and a third output port,
wherein:
the battery is in communication with the fifth input port;
the sixth input port is in communication with the first output port and the second output port; and
the third output port is in communication with the second output port.
